(19) United States
(12) Patent Application Publication
(10) Pub. No.: US 2024/0001260 A1
(43) Pub. Date: Jan. 4, 2024

(54) APPARATUS FOR BIO-PRODUCT PROCESSING

(71) Applicant: CYTIVA SWEDEN AB, Uppsala (SE)

(72) Inventors: Stefan K. Eriksson, Uppsala (SE); Andreas Bergstrom, Uppsala (SE); Andreas Sjolund, Uppsala (SE); Gustav D. Andersson, Uppsala (SE); Mikael C. Sjogren, Uppsala (SE)

(21) Appl. No.: 18/252,331

(22) PCT Filed: Nov. 17, 2021
(86) PCT No.: PCT/EP2021/082008

§ 371 Date: May 9, 2023

(30) Foreign Application Priority Data
Nov. 23, 2020 (GB) 2018386.9

(51) Int. Cl. B01D 15/22; C12M 1/00; G01N 30/60; C12M 3/00

(52) U.S. Cl. CPC B01D 15/22; C12M 47/12; G01N 30/6052; C12M 23/48; C12M 23/52

(57) ABSTRACT

The present invention relates to an apparatus (200) for bio-product processing. The apparatus (200) comprise a processing column (201) comprising an upper column tube (202B) releasably coupled to a lower column tube (202A). The lower column tube (202A) has a processing chamber (218) provided therein. The apparatus (200) additionally comprises an adapter (222) for moving within the processing column (201) so as to vary the volume of the processing chamber (218) within the lower column tube (202A), and a column stand (214) for supporting the adapter (222).

### FIELD

The present invention relates generally to an apparatus for bio-product processing. More specifically, the present invention relates to an improved apparatus that incorporates a processing column that provides a processing chamber for the production, processing and/or separation etc. of bio-products. Such bio-products may include, for example, RNA for use in personalised medicine, bio-pharmaceutical products separated from cell cultures, etc.

### BACKGROUND

Various apparatus is known for producing or processing biopharmaceutical products and components thereof; biologically related/derived products used in various processes and for research supplies; etc. Many such desired end bio-products are produced or processed using apparatus in a processing chamber that is formed in a processing column.

For example, an adjustable Oligo column, commercially available from Cytiva Life Sciences™, can be used for oligonucleotide synthesis, and is designed for process development with an ÄKTA™ OligoPilot™ plus synthesizer.

Other column based devices, for example as may be used for chromatography based bio-product/bioprocessing separation systems, are also known. See, for example, the patent applications WO 2013/191628 A1 and WO 2015/088427 A1, the contents of which are hereby incorporated herein by reference to the maximum permissible extent.

However, whilst various adjustable column devices have been in use for a long time, and the quality of the Cytiva Life Sciences™ adjustable Oligo column in particular is high and the product well-established, it is desirable to provide an even further improved apparatus that is safer to operate, easier to use and to maintain, and which has an increased potential production capability.

Hence, the present invention, as defined by the appended claims, is provided.

### SUMMARY

In a first aspect, the present invention thus relates to an apparatus for bio-product processing. The apparatus includes a processing column comprising an upper column tube releasably coupled to a lower column tube having a processing chamber provided therein. An adapter for moving within the processing column so as to vary the volume and/or bed height of the processing chamber within the lower column tube and a column stand for supporting the adapter are also provided.

In further aspects of the invention, a column processing module for an apparatus comprising a lower column tube providing a process chamber for retaining a bio-product therein and a support for retaining the lower column tube in a substantially level position, and a lower column tube for use in the apparatus are provided.

By providing a multiple-part column design with a column stand and adapter, an apparatus is provided that is easier to clean and maintain. Such a design also enables part of a processing column to be removed for cleaning or extracting product therefrom whilst a separate replacement column part is installed. Hence, up-time for processing/production can be increased by constantly swapping column processing modules, which may themselves optionally be pre-packaged with various sterile materials that are needed for the processing/production.

Another benefit of various aspects and embodiments of the present invention is that removal and/or replacement of various parts of the apparatus can be made to be hoist-free, such that a compact/small footprint and easy to use/maintain apparatus is provided.

### BRIEF DESCRIPTION OF THE DRAWINGS

FIG. 1 shows a column-based device for a chromatography separation process in accordance with the prior art;

FIG. 2 shows the column-based device of FIG. 1 having an adapter provided therein, in accordance with the prior art;

FIGS. 3A and 3B show an apparatus for bio-product processing in accordance with an embodiment of the present invention;

FIGS. 4A, 4B and 4C show use of the apparatus of FIGS. 3A and 3B during a column process running mode;

FIGS. 5A, 5B and 5C show use of the apparatus of FIGS. 3A and 3B during a bottom disconnection operation;

FIGS. 6A, 6B and 6C show use of the apparatus of FIGS. 3A and 3B during a top disconnection operation; and

FIGS. 7A, 7B and 7C show a column processing module for use in the apparatus of FIGS. 3A and 3B in accordance with an embodiment of the present invention.

### DETAILED DESCRIPTION

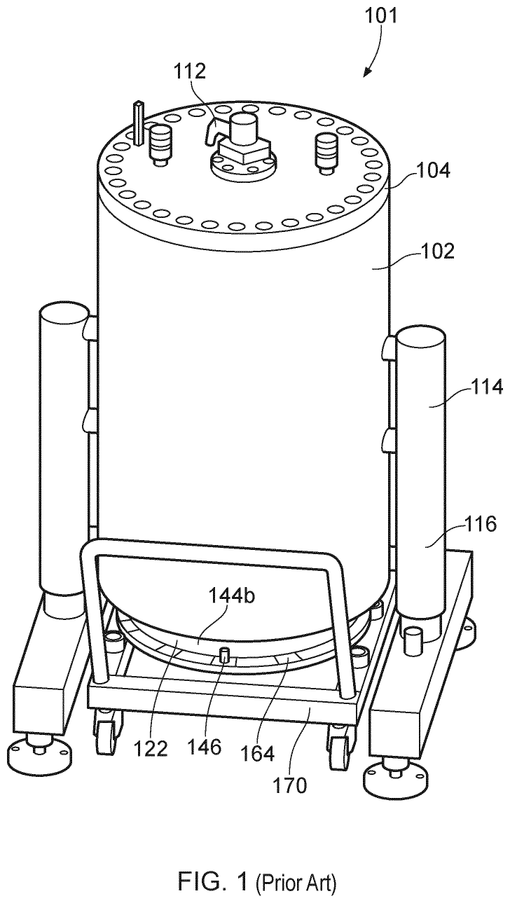

FIG. 1 shows a column-based device 101 for a chromatography separation process in accordance with the prior art. A tubular housing 102 is provided in which a fluid space 118 and a bed space 120 are defined. An adapter 122 is further provided for providing a bed 124 of packed particulate media in the bed space 120.

The tubular housing 102, a top end-piece 104 and a bottom end-piece 106 are secured together to form a fluid tight seal by means of tension bolts. The tubular housing 102 and top and bottom end-pieces 104, 106 are typically composed of a solvent resistant material such as stainless steel or a high-strength plastic material such as polypropylene. Where the column-based device 101 is to be used for the separation of biologically active substances, the material is biologically inert such that it does not elicit an immune response in humans. An adapter rod 110 extends through an opening 112 in the top end-piece 104 and into the tubular housing 102. The column-based device 101 is also arranged on a frame 114 provided with legs 116, so that the column-based device 101 may be placed on a floor in a stable position.

The column-based device 101 is shown where the bottom end piece 106 has been removed and the adapter 122 has been lowered to a position where a part of the adapter 122 is still within the tubular housing 102 of the column-based device 101, so that compression fluid held within the column-based device 101 above the adapter 122 cannot escape therefrom.

In this lowered position a first sliding ring 144a of the adapter 122 is positioned to rest on an inner surface of the tubular housing 102 in order to provide a tight seal between the adapter 122 and the tubular housing 102. In the first lowered adapter position, cavities 146 arranged in a periphery of the adapter 122 are positioned outside of the tubular housing 102, so that it is possible to get access to the cavities 146 from outside of the column-based device 101 after removing a second sliding ring 144b, which covers the cavities 146.

The cavities 146 are arranged closer to an end surface 141 of the adapter 122 than to a top surface 143 thereof. Fastening means (not shown) may be released through the cavities 146 for removing a filter from the adapter 122. A support trolley 170 is arranged under the adapter 122, so that the filter may rest on the trolley 170 when the filter is removed from the adapter 122.

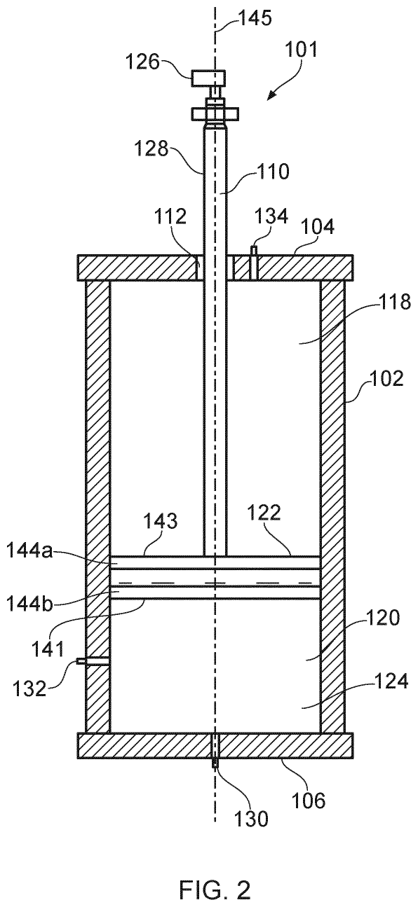

FIG. 2 shows cross-sectional view of the column-based device 101 of FIG. 1 having an adapter 122 provided therein, in accordance with the prior art. The adapter 122 is used together with a filter (not shown) and a distributor plate (not shown) for packing a bed of packing material within the tubular housing 102. The distributor plate distributes fluid within the column-based device 101 and the filter prevents particles from the bed of packing material entering into holes or openings within the distributor plate, such that the filter prevents the particles from escaping.

The tubular housing 102 and the end pieces 104, 106 form a fluid space 118 and bed space 120, which spaces 118, 120 both are fluid tight and capable of withstanding high operating pressures. A wide range of column capacities is also possible, typically ranging from 0.1 to 2000 litres. The bed space 120 is defined by the tubular housing 102, the bottom end-piece 106 and the adapter 122 connected to the adapter rod 110.

The bed space 120 is filled with a bed 124 of packing material, which is usually particulate in nature and consists of a porous medium. A liquid mobile phase is arranged to enter through an inlet 126 at the end of the adapter rod 110 which flows through a central channel 128 in the adapter rod 110 and further to the adapter 122. The liquid mobile phase thereafter moves through the bed 124 of packing material and is finally removed via an outlet 130 provided in the bottom end-piece 106. Typically, the porous medium enclosed in the column 101 as a packed bed 124 is generally formed by consolidating a suspension of discrete particles, known as slurry, that is pumped, poured or sucked into the column-based device 101 from a bore or nozzle 132 located within the tubular housing 102.

The bed 124 of packed particulate medium is obtained by the downward movement of the adapter 122 to compress the bed 124 between the adapter 122 and the bottom end-piece 106. The compression force and downward movement of the adapter 122 is achieved by a providing a pressurized fluid into the fluid space 118 above the adapter 122. The fluid, e.g. water, is pumped into the fluid space 118 through a valve 134 located within the top end-piece 104.

Such a prior art column-based device 101 provides for improved maintenance on chromatography columns, especially for those used in large volume industrial-scale chromatography, by reducing the need to use heavy lifting equipment, such as hoists or cranes, to dismantle the columns. However, such a process is still relatively slow and labour intensive since it requires the removal of the bottom end-piece 106, and hence is generally best suited to infrequently required maintenance operations such as replacing filters/seals etc. of the adapter 122.

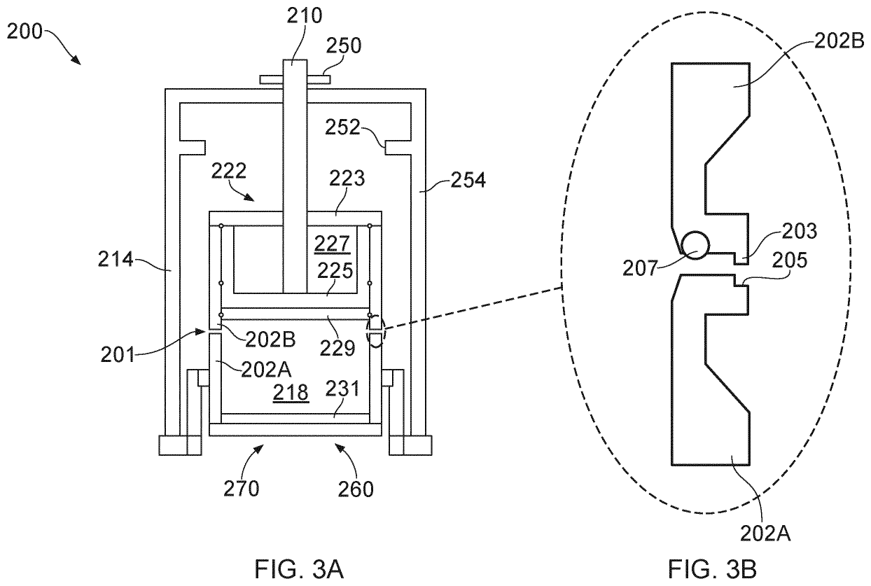

FIGS. 3A and 3B show an apparatus 200 for bio-product processing in accordance with an embodiment of the present invention.

FIG. 3A shows a schematic of the apparatus 200 in cross-sectional view. The apparatus 200 incorporates a processing column 201 comprising an upper column tube 202B that is releasably coupled to a lower column tube 202A by way of the mechanism described below in connection with FIG. 3B.

The lower column tube 202A is coupled to a chamber base plate 231 and defines a processing chamber 218 therein. The processing chamber 218 may be used for producing and/or processing various bio-products. For example, DNA/RNA/mAb related products etc. Such bio-products may also be processed/produced in generally sterile/aseptic conditions.

The apparatus 200 further includes an adapter 222 for moving within the processing column 201 so as to vary the volume of the processing chamber 218 within the lower column tube 202A. The adapter 222 includes an adapter rod 210 that is connected to a lower chamber casing 225. The lower chamber casing 225 supports a flow distribution/filter plate 229. The adapter rod 210 passes through a sealing arrangement provided in an opening in an upper plate 223, such that the adapter rod 210 can slide through the opening. An outer periphery of the upper plate 223 is releasably coupled to an upper end of the upper column tube 202B in a fluid-tight arrangement. Together the upper plate 223 and the lower chamber casing 225 define a fluid-tight hydraulic chamber 227 therebetween.

Hydraulic fluid (e.g. pressurised water) may thus be introduced into and/or removed from the hydraulic chamber 227 through a port (not shown) provided in the upper plate 223 in order to move the lower chamber casing 225 to a desired position within the processing column 201. The volume of the processing chamber 218 can be thus be adjusted to a required volume by using the column adapter 222, to account for a particular support used, the operational scale desired, etc. For certain applications, a variable bed height within the processing chamber 218 may thus also be provided so as to enable processing scale variation, etc.

The apparatus 200 is further provided with a column stand 214 for supporting the adapter 222. The column stand 214 includes a frame 254 that generally surrounds the processing column 201. Optionally, the frame 254 may provide additional support for the upper column tube 202B, e.g. by way of a locking mechanism, bolts, a support bracket, etc. An axially aligned centred through-hole is provided in an upper part of the frame 254 to enable the adapter rod 210 of the adapter 222 to pass therethrough. A rod locking mechanism 250 is provided adjacent to the through-hole of the frame 254. The rod locking mechanism 250 is operable to secure the adapter rod 210 in various positions with respect to the frame 254. The rod locking mechanism 250 may, for example, incorporate a brake mechanism, or the like, which can be either manually and/or automatically actuated.

Within the frame 254 a top plate holder 252 is provided. The top plate holder 252 optionally comprises a coupling mechanism (not shown) for connecting to the upper plate 223 when it is released from the upper end of the upper column tube 202B.

As is also shown in FIG. 3A, the lower column tube 202A is provided as part of a column processing module 260 upon a support 270. The support is provided for retaining the lower column tube 202A in a substantially level position during use of the apparatus 200. The support 270 preferably, but not necessarily, connects to a plurality of wheels 262 that enable the column processing module 260 to be easily moved on a floor surface, e.g. of a bio-processing or production facility.

Such a column processing module 260 can thus readily be removed from the apparatus 200 for further processing and/or cleaning and replaced by a similar column processing module 260, e.g. that is pre-packed with various materials, components, resins, beads, etc. Hence, bio-processing can be speeded up by using multiple column processing modules so as to provide a higher production through-put when using various embodiments of the present invention.

FIG. 3B shows an expanded view of a releasable connection for coupling the upper column tube 202B to the lower column tube 202A. The upper column tube 202B comprises a radially outwardly disposed circumferential notch 203. The lower column tube 202A comprises a radially outwardly disposed circumferential groove 205 for receiving the circumferential notch 203 therein. Such an arrangement allows for accurate and stable alignment of the upper and lower column tubes 202A, 202B.

The upper column tube 202B additionally comprises a radially inwardly disposed circumferential O-ring seal 207. The weight of the upper column tube 202B urges the O-ring seal 207 into a sealing engagement with an upper end-portion surface of the lower column tube 202A when the upper and lower column tubes 202A, 202B are in a stacked arrangement.

Various other releasable connections for coupling the upper column tube 202B to the lower column tube 202A will also be apparent to those skilled in the art. For example, the upper column tube 202B may be provided with a notch and the lower column tube 202A a groove, multiple grooves/notches etc. may be provided at a variety of positions, one or more sealing arrangements/components may be provided at various positions on one or more of the upper and/or lower column tubes, etc.

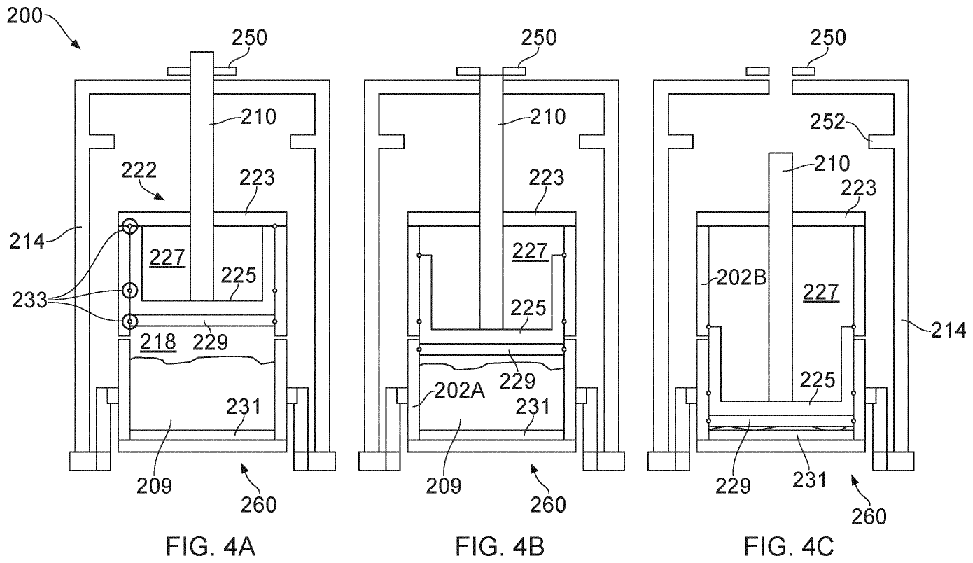

FIGS. 4A, 4B and 4C show use of the apparatus 200 of FIGS. 3A and 3B during a column process running mode.

FIG. 4A shows the apparatus 200 in a first operating position. The lower chamber casing 225 is sealed within the upper column tube 202B by upper and lower O-rings 233, and is in a position that is generally adjacent to the upper plate 223. A further O-ring 233 also initially seals a circumferential periphery of the flow distribution/filter plate 229 within the upper column tube 202B. Provision of such O-rings 233 allows movement of the adapter 222 over a soft joint provided between the upper and lower column tubes 202A, 202B without there being any substantial leakage. The column processing module 260 is further provided with a bio-product 209 within the process chamber 218, and the rod locking mechanism 250 is in an unlocked state.

In various embodiments, a slurry inlet may be provided in the upper column tube 202B. A slurry inlet/outlet may also be provided in a lower region of the lower column tube 202A.

FIG. 4B shows the apparatus 200 in a second operating position. In the second operating position hydraulic fluid is introduced into the hydraulic chamber 227 through an inlet or valve (not shown) provided in the upper plate 223. A further outlet or valve (not shown) may also be provided in the upper plate 223. The hydraulic fluid causes the hydraulic chamber 227 to expand forcing the lower chamber casing 225 with the flow distribution/filter plate 229 attached thereon away from the upper plate 223 towards the lower column tube 202A.

FIG. 4C shows the apparatus 200 in a third and final operating position. In the final operating position, the lower O-ring 233 of the chamber casing 225 and the O-ring 233 provided with the flow distribution/filter plate 229 have been pushed into the lower column tube 202A. The flow distribution/filter plate 229 and the chamber base late 231 thus end up in close proximity. Further, as the chamber casing 225 is pushed into the process chamber 218, a valve (not shown) therein is opened to allow the bio-product 209 to be extracted. In various embodiments, the bio-product 209 may alternatively be extracted through a slurry outlet.

In operation, for example during an Oligo process, the upper and lower O-rings 233 provided in the lower chamber casing 225 do not always necessarily move over the joint between the upper and lower column tubes 202A, 202B during the process running. For such processes (e.g. used when making peptides, synthetic molecules, etc.) a molecular chain may be built up on a substrate, such as beads. As these molecular chains grow, they take up increased space and so there is a need to increase volume/maintain pressure in the processing chamber 218. The apparatus 200 can thus be adjusted to account for such volume/pressure requirements, and thus may not need to follow all of the steps depicted in FIGS. 4A-C.

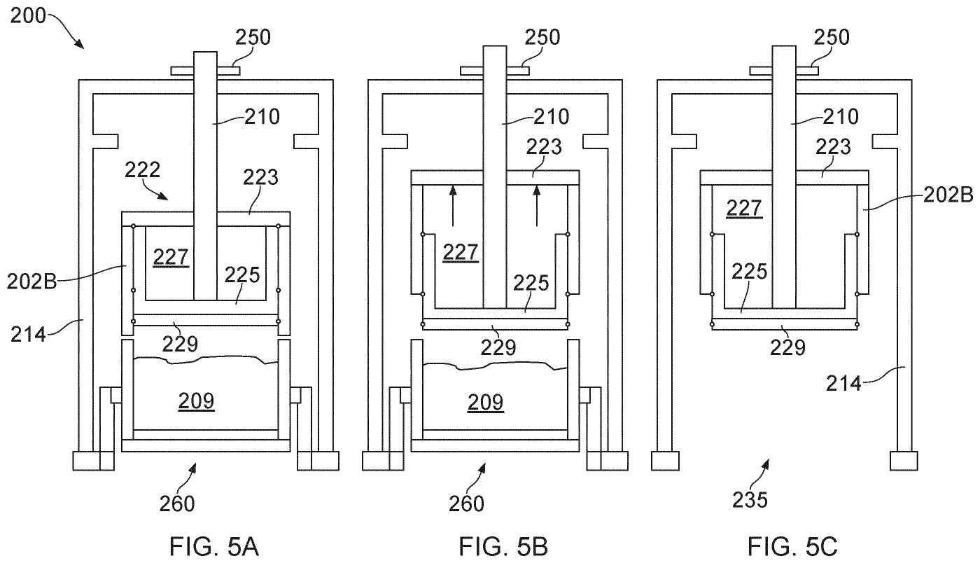

FIGS. 5A, 5B and 5C show use of the apparatus 200 of FIGS. 3A and 3B during a bottom disconnection operation.

FIG. 5A shows the apparatus 200 in the first operating position. The lower chamber casing 225 is sealed within the upper column tube 202B by the upper and lower O-rings 233, and is in a position that is generally adjacent to the upper plate 223. The further O-ring 233 also initially seals a circumferential periphery of the flow distribution/filter plate 229 within the upper column tube 202B. The column processing module 260 is shown provided with a bio-product 209 within the process chamber 218, and the rod locking mechanism 250 is in an initial unlocked state.

FIG. 5B shows the apparatus 200 in a second operating configuration. In the second operating configuration the rod locking mechanism 250 is engaged such that the adapter rod 210 is immobilised with respect to the column stand 214. The rod locking mechanism 250 may be manually engaged and/or automatically engaged (e.g. by use of solenoid or hydraulic actuators) as part of a remotely controlled operation.

Once the rod locking mechanism 250 is engaged, hydraulic fluid is introduced into the hydraulic chamber 227. Introduction of the hydraulic fluid into the hydraulic chamber 227 causes the upper plate 223 to lift towards the rod locking mechanism 250. Since the upper plate 223 is connected to the upper column tube 202B, the latter also lifts, thereby causing the upper column tube 202B to disengage from the lower column tube 202A. The lower column tube 202A thereby remains in situ in the column processing module 260, whilst the upper column tube 202B is moved clear thereof in a vertical direction.

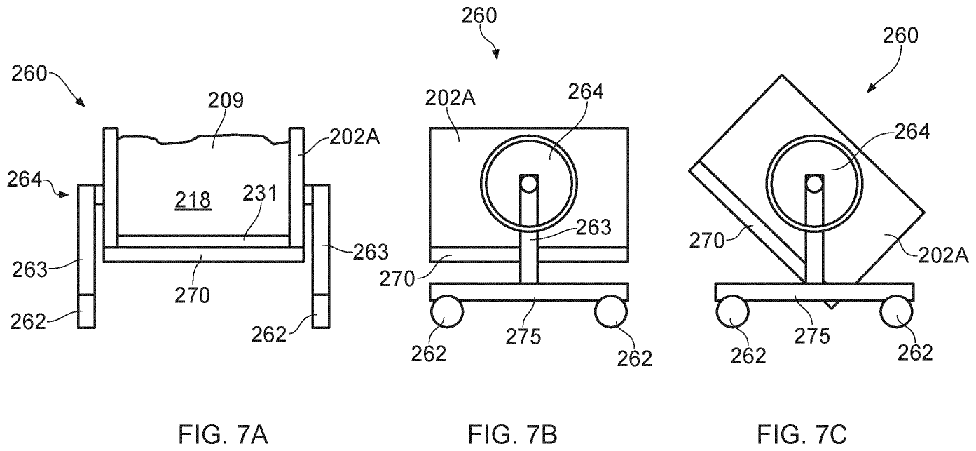

FIG. 5C shows the apparatus 200 in a third operating configuration. In the third operating configuration, whilst the rod locking mechanism 250 remains engaged, the column processing module 260 has been removed from within a lower volume portion 235 defined by the column stand 214. The column processing module 260 is preferably, but not necessarily, of the type shown in FIGS. 7A to 7C (see below) that is provided with a plurality of wheels 262 provided thereon to aid when moving the column processing module 260. In this third operating configuration, the flow distribution/filter plate 229 is also readily accessible, such that cleaning/maintenance etc. thereof can more easily be performed.

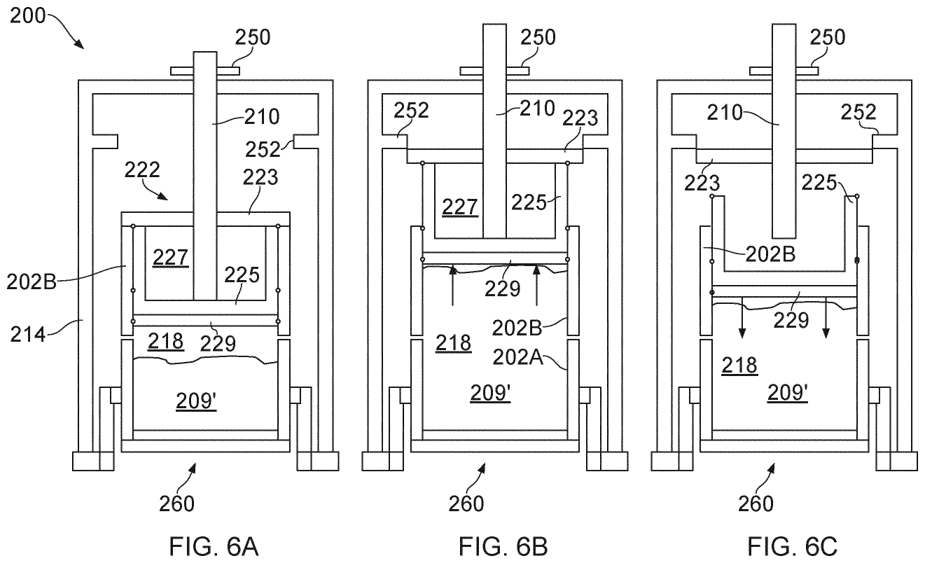

FIGS. 6A, 6B and 6C show use of the apparatus 200 of FIGS. 3A and 3B during a top disconnection operation.

FIG. 6A shows the apparatus 200 in the first operating position. The lower chamber casing 225 is sealed within the upper column tube 202B by the upper and lower O-rings 233, and is in a position that is generally adjacent to the upper plate 223. The further O-ring 233 also initially seals a circumferential periphery of the flow distribution/filter plate 229 within the upper column tube 202B. The column processing module 260 is shown provided with a bio-product replacement fluid 209′ within the process chamber 218, and the rod locking mechanism 250 in an initial unlocked state.

FIG. 6B shows the apparatus 200 in a second operating state. In the second operating state, the upper plate 223 is first disconnected from the upper column tube 202B. Then the process chamber 218 is filled with bio-product replacement fluid 209′ and the whole of the adapter 222 is lifted. Once the upper plate 223 is in position adjacent to the top plate holder 252, rod locking mechanism 250 is engaged to prevent the adapter rod 210 from moving relative to the column stand 214. Optionally, top plate holder 252 may comprise a mechanism for releasably securing the upper plate 223 thereto so as to prevent the upper plate 223 from dropping should the rod locking mechanism 250 fail or be accidentally disengaged. Additionally, or alternatively, various bolts, releasable fasteners, etc. may be used to secure the upper plate 223 to the top plate holder 252.

FIG. 6C shows the apparatus 200 in a third operating state. In the third operating state bio-product replacement fluid 209′ is drained from the process chamber 218 and the lower chamber casing 225, and the flow distribution/filter plate 229 connected thereto, descend into the upper column tube 202B. Since the rod locking mechanism 250 is still engaged, the adapter rod 210 remains in situ. Upper plate 223 remains engaged with the top plate holder 252 within an upper volume of the column stand. Hence, in this state, the upper plate 223, the inside of the lower chamber casing 225, and a top portion of the adapter 222 are made available for maintenance.

FIGS. 7A, 7B and 7C show a column processing module 260 for use in the apparatus 200 of FIGS. 3A and 3B in accordance with various embodiments of the present invention.

FIG. 7A shows a sectional view of the column processing module 260. The column processing module 260 includes a lower column tube 202A and a chamber base plate 231 that together define a process chamber 218. The process chamber 218 may comprise one or more bio-processing materials for processing or producing bio-products. Such materials may further be supplied already packed/pre-packaged therein, optionally compacted and/or in an already sterilised/aseptic state.

Both the lower column tube 202A and a chamber base plate 231 are connected to a support 270 that can be used to maintain the lower column tube 202A and/or chamber base plate 231 in a substantially level position. Additionally, the support 270 is rotatably coupled, via at least one tilt mechanism 264, to first and second supports 263 provided on a chassis 275.

The chassis 275 further incorporates a plurality of wheels 262 that enable the column processing module 260 to be readily moved on a floor surface, either with or without there being any contents in the processing chamber 218.

FIG. 7B shows a side view of the column processing module 260 with the lower column tube 202A and the chamber base plate 231 in a substantially level position. In this position, one or more tilt mechanism 264 may be in a locked position to maintain that level position as the column processing module 260 is moved.

FIG. 7C shows a side view of the column processing module 260 in a tilted state. If locking is provided, one or more tilt mechanism 264 may be unlocked before the support 270, with the lower column tube 202A and chamber base plate 231 supported thereon, is rotated away from the level position.

The lower column tube 202A may optionally be unpacked thorough a side port (not shown) provided therein and/or manually. This specific embodiment also allows the lower column tube 202A to be rotated so as to assist in pouring the contents thereof out.

Whilst various preferred embodiments may use a tilt mechanism therein, those skilled in the art would be aware that the invention is not limited only thereto. For example, various column processing module designs would be apparent, including those having substantially fixed orientations for the processing chambers thereof.

Various embodiments of the present invention may thus be provided that are easier to clean/maintain, that have process chambers that are easier to transport/swap in use, which permit a hoist-free operation, that enable efficient interchange of parts of an apparatus so one can be cleaned or serviced whilst another replaces it, which employ flow-through reactor technology, which provide convenient packing/pre-packing of a solid support material, which are solvent resistant, which have a simple and robust design and/or which maximise the “up-time” use of a bio-production apparatus, etc.

Where large product systems/bioprocessing systems (e.g. having a capacity of 1000 litres, 2000 litres etc.) are employed, use of a hoist free embodiment further improves operational safety aspects by avoiding the need to hoist heavy free-swinging components, such as reactor lids, etc. Safe plant operation is further enhanced where any dangerous chemicals might remain after a processing operation, since use of a shorter, mobile, column portion permits easier removal of waste/dangerous products, it thereby being easier to access the bottom of a shorter column portion with, for example, a spatula, vacuum aspirator, etc.

Those skilled in the art will also realise that many materials may be used to construct various components or parts of an apparatus provided in accordance with the present invention. For example, various solvent resistant materials, stainless steel materials, and/or plastic materials, such as polypropylene, etc., may be used as appropriate for any desired end application.

Various aspects and embodiments of the present invention have been described herein. Nevertheless, those skilled in the art would be aware that various modifications may be made that would still embody the inventive concept(s) envisaged. For example, the skilled person would realise that various components of both the prior art and the embodiments described herein may be combined to yield yet further embodiments of the present invention.

Furthermore, it is hereby noted that all hereinbefore-mentioned patents, patent applications and/or specified products that are commercially available are hereby incorporated by reference in their entirety where such practice is allowed.

1. An apparatus for bio-product processing, comprising:
a processing column comprising an upper column tube releasably coupled to a lower column tube having a processing chamber provided therein;
an adapter for moving within the processing column so as to vary the volume of the processing chamber within the lower column tube; and
a column stand for supporting the adapter.

2. The apparatus according to claim 1, wherein the column stand comprises a frame.

3. The apparatus according to claim 2, wherein the frame comprises a top plate holder.

4. The apparatus according to claim 1, comprising a rod locking mechanism for retaining the adapter in a raised position.

5. The apparatus according to claim 1, wherein the lower column tube is provided as part of a column processing module upon a support for retaining the lower column tube in a substantially level position during use of the apparatus.

6. The apparatus according to claim 5, wherein the column processing module further comprises a wheel or a plurality of wheels that enable the column processing module to be moved on a floor surface and/or at least one tilt mechanism for moving the lower column tube to and from said substantially level position.

7. The apparatus according to claim 1, wherein the adapter is hydraulically actuatable and comprises an adapter rod that is removably connectable to a lower chamber casing through an opening provided in an upper plate, such that the adapter rod can slide through the opening, wherein an outer periphery of the upper plate is releasably couplable to an upper end of the upper column tube, and wherein the upper plate and the lower chamber casing define a fluid-tight hydraulic chamber therebetween.

8. A column processing module for an apparatus, comprising a lower column tube providing a process chamber for retaining a bio-product therein and a support for retaining the lower column tube in a substantially level position.

9. The column processing module according to claim 8, further comprising at least one wheel to enable the column processing module to be moved on a floor surface.

10. The column processing module according to claim 8, further comprising at least one tilt mechanism for moving the lower column tube to and from said substantially level position.

11. The column processing module according to claim 8, further comprising one or more bio-processing materials for processing and/or producing bio-products pre-packaged therein.

12. The column processing module according to claim 8, wherein at least part of the column processing module is supplied in a sterilised/aseptic state.

13. A lower column tube for use in the apparatus of claim 1, having a process chamber for retaining a bio-product provided therein.

14. The lower column tube according to claim 13, further comprising one or more bio-processing materials for processing and/or producing bio-products pre-packaged in the process chamber.

15. The lower column tube according to claim 13, wherein the lower column tube is in a sterilised/aseptic state.
